(19) United States
(12) Patent Application Publication
(10) Pub. No.: US 2006/0074010 A1
(43) Pub. Date: Apr. 6, 2006

(54) METHOD AND APPARATUS FOR PURIFYING PHARMACEUTICAL COMPOSITIONS USING SUPERCRITICAL FLUIDS

(75) Inventors: Pratibhash Chattopadhyay, North Royalton, OH (US); Boris Y. Shekunov, Aurora, OH (US)

Correspondence Address: RANKIN, HILL, PORTER & CLARK, LLP, 925 EUCLID AVENUE, SUITE 700, CLEVELAND, OH, 44115-1405, US

(73) Assignee: FERRO CORPORATION, Cleveland, OH (US)

(21) Appl. No.: 11/162,698

(22) Filed: Sep. 20, 2005

Related U.S. Application Data
(60) Provisional application No. 60/615,153, filed on Oct. 1, 2004.

(51) Int. Cl. A61K 38/00; C07K 14/00

(52) U.S. Cl. 514/2; 514/169; 530/412

(57) ABSTRACT

The present invention provides a method and an apparatus for removing impurities from biodegradable polymers and/or pharmaceutical compounds. In accordance with the method, a stream of supercritical fluid and, optionally, a modifying co-solvent, is used to dissolve the impurities. The pressure and temperature of the supercritical fluid or supercritical fluid/co-solvent mixture is controlled such that the supercritical fluid or mixture acts as a solvent for the impurities, but not for the biodegradable polymers and/or pharmaceutical compounds.

### BACKGROUND OF THE INVENTION

1. Field of Invention

The present invention provides a method and an apparatus for removing impurities from pharmaceutical compounds, ingredients, materials and products using supercritical fluids.

2. Description of Related Art

It is essential that pharmaceutical compositions be purified before they are delivered into the human body. The presence of impurities such as residual monomers, unreacted reagents and residual organic solvents in such compositions can lead to a reduction in the efficacy of the pharmaceutical compositions, can present toxicity concerns and can cause undesirable side effects and/or other serious complications. Furthermore, impurities can also reduce the shelf life of pharmaceutical compositions, which leads to storage and packaging problems.

The synthesis of pharmaceutical compositions in most cases is facilitated through the use of organic solvents, catalysts, monomers and other reactive agents that may be harmful to the human body at high levels. Often times, residual amounts of these materials can remain in the final pharmaceutical compositions as impurities, which makes them unsuitable and potentially hazardous for administration to humans. A significant amount of effort is therefore taken to ensure the purity of such products. In many instances, the processes employed to purify pharmaceutical compositions are elaborate and cost intensive, but not necessarily successful in completely eliminating impurities. The problem of residual impurities in pharmaceutical compositions is exacerbated when multiple impurities are present.

Conventional methods of purifying pharmaceutical compositions include high temperature treatment, vacuum drying and solvent extraction using non-toxic solvents. High temperature treatment and/or vacuum drying are typically used to remove volatile impurities such as organic solvents. Non-volatile impurities are typically removed by washing with copious amounts of non-toxic solvents followed by heat treatment and/or vacuum drying to remove the washing solvent.

These conventional techniques often require large processing times at high cost and present scale up complexities. In addition, the thermal treatment process, for example, is unsuitable for purifying thermally labile pharmaceutical compositions. The vacuum drying technique is particularly slow and uneconomical due to the high costs involved in pumping and high scale up costs. The solvent extraction technique is time consuming, cost intensive and generates large waste streams. Apart from these disadvantages, these techniques often do not sufficiently provide the levels of purification required for the processing of acceptable pharmaceutical compositions.

### BRIEF SUMMARY OF THE INVENTION

The present invention provides a method and an apparatus for purifying pharmaceutical compositions, including compounds, ingredients, materials and products used in medicinal and therapeutic preparations for administration to humans. The method according to the invention utilizes supercritical fluid (“SCF”) as a purifying extraction agent. The primary property of SCF that makes it suitable for use in the invention is that its solvent power for dissolving various materials can be controlled by changing its density, which can be controlled by manipulating its pressure, temperature and by the addition of modifying co-solvents. The method of the present invention provides effective and environmentally benign purifying of pharmaceutical compositions at mild operating conditions, at relatively low cost with minimum scale up complexities. Furthermore, the method of the invention also offers a one step purifying process whereby multiple impurities can be removed by changing the density or the solvent properties of the SCF employed.

The removal of impurities from pharmaceutical compositions according to the invention is preferably accomplished using a continuous supercritical fluid extraction process. The pharmaceutical composition is first loaded into a high-pressure extraction chamber. SCF is introduced into the high-pressure extraction chamber, either with or without the addition of modifying co-solvents, as a continuous flow at a pressure and temperature whereby it dissolves the impurity or impurities present in the pharmaceutical composition, but not the biologically active pharmaceutical component itself. The impurity laden SCF continuously exits the extraction vessel. The exit stream from the extraction column is continuously analyzed using a detection system in order to monitor and measure the concentration of impurity or impurities present. When the pharmaceutical material is sufficiently purified, the flow of pure SCF can be continued to remove any traces of modifying co-solvent, but then is stopped and the extraction column is depressurized and the purified pharmaceutical composition is subsequently collected.

In a first embodiment of the invention, the pharmaceutical composition is in the form of a particulate packed or fluidized bed inside the extraction column and the SCF is passed through the pharmaceutical composition at a constant flow rate, pressure and temperature in order to extract impurities. In a second embodiment of the invention, the pharmaceutical material is placed on perforated trays present in the extraction column. Impurities are extracted by flowing SCF through the pores of these trays at a constant flow rate, pressure and temperature. In both embodiments, the operational pressure and temperature conditions are selected such that the impurity present in the pharmaceutical composition is soluble in the SCF, but the biologically active component is not. If the pharmaceutical composition contains multiple impurities, the operational extraction conditions can be changed in order to eliminate each one or portions of the impurities separately. In both embodiments of the invention, the purification of pharmaceutical compositions is advantageously carried out in one step at mild operating conditions without the use of any potentially toxic organic solvents.

The foregoing and other features of the invention are hereinafter more fully described and particularly pointed out in the claims, the following description setting forth in detail certain illustrative embodiments of the invention, these being indicative however, of but a few of the various ways in which the principles of the present invention may be employed.

### BRIEF DESCRIPTION OF THE DRAWINGS

FIG. 1 is a schematic representation of an apparatus used to purify pharmaceutical compositions in accordance with a first embodiment of the invention.

FIG. 2 is a schematic representation of an apparatus used to purify pharmaceutical compositions in accordance with a second embodiment of the invention.

### DETAILED DESCRIPTION OF THE INVENTION

In accordance with the present invention, impurities are removed from pharmaceutical compositions by extracting impurities using a stream of SCF or a mixture comprising a SCF and one or more modifying co-solvents. The solvent property of the SCF or mixture can be tuned by adjusting its pressure, temperature and co-solvent concentration in order to facilitate maximum dissolution of the impurity in the SCF. The SCF or SCF/co-solvent mixture diffuses into pores and/or crystal lattice of a solids portion of the pharmaceutical composition and extracts the impurities from the pharmaceutical composition, thereby purifying it.

Although optional, co-solvents can be added to the SCF to increase the solubilization power of the mixture. The use of a modifying co-solvent may be required for efficient removal of some solid impurities or liquid impurities, which are poorly miscible with pure SCF. These additional co-solvents may comprise any organic solvents that are miscible or at least partially miscible with SCF such as ethanol, methanol, acetone and others. The resulting mixture preferably remains in the supercritical homogeneous phase so that the preferred content of organic solvent in SCF is below 20% mole fraction and the mixture is above its critical pressure, that is the pressure where the mixture becomes a single homogeneous phase.

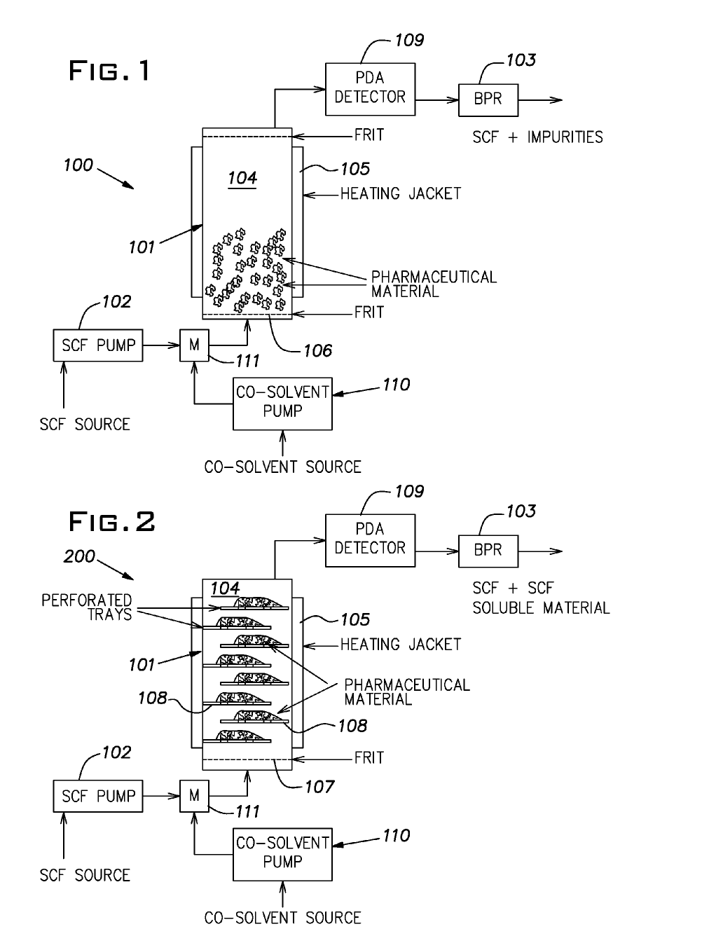

A system or apparatus 100 for implementing a method according to the first embodiment of the invention is shown in FIG. 1. The apparatus 100 comprises a high-pressure extraction vessel 101, a SCF pump 102, a backpressure regulator 103 and a photo diode array (PDA) detector 109. The high-pressure extraction vessel 101 is preferably tubular in shape such that it defines an axis, and has first and second ends that are spaced axially apart. The high-pressure extraction vessel 101 has an inner surface that defines an extraction chamber 104. The extraction vessel must be provided with means (not shown) for accessing the interior of the vessel. The extraction vessel should be equipped with an electrical or circulating heating jacket 105 to maintain the extraction vessel under isothermal conditions. An additional liquid high-pressure pump 110 can be used to supply organic solvents to modify the SCF.

A pharmaceutical composition is loaded into the extraction chamber 104. The pharmaceutical composition is preferably in the form of discrete particles. Alternatively, the pharmaceutical composition can be in the form of one or more large pieces. The pharmaceutical composition preferably comprises a biologically active material, for example a drug, a pharmaceutical, or a therapeutic agent, and at least a first impurity. The pharmaceutical composition can further comprise medicinal agents, proteins, peptides, sugars, toxins, viral materials, diagnostic aids, nutritional materials, alkaloids, alkyloids, animal and/or plant extracts, dyes, polymer precursors, antigens, enzymes, catalysts, nucleic acids, zeolites and combinations thereof.

In most cases, the pharmaceutical composition will further comprise a material selected from the group consisting of carriers, polymers, fillers, disintegrants, binders, solubilizers, excipients and combinations thereof. The preferred pharmaceutical compositions comprise a biologically active material, a biodegradable polymer and at least one impurity. Preferred biodegradable polymers include, for example, polysaccharides, polyesters, polyethers, polyanhydrides, polyglycolides, polylactic acids and their copolymers, polycaprolactones, polyethylene glycols and polypeptides.

The SCF pump 102 is preferably a P-200 high-pressure reciprocating pump, which is commercially available from Thar Technologies, Inc. (Pittsburgh, Pa.). Suitable alternative pumps include diaphragm pumps and air-actuated pumps that provide a continuous flow of SCF. The high-pressure pump preferably comes factory-equipped with a burst-type rupture disc, manufactured by Fike Inc. (Blue Springs, Mo.), which is plumbed into a pressure relief system.

The SCF 102 pump is in fluid communication with the liquid pump 110 through a mixing unit 111, which can be a T-piece connector or static mixer that can provide a fast and homogenous mixing between the SCF and the modifying co-solvent. The flow ratio between the SCF and the liquid pumps defines the mole composition of the mixed fluid.

The liquid solvent pump is preferably a semi-preparatory high-pressure liquid chromatography (HPLC) reciprocating pump, such as the model PU-2080, which is commercially available from Jasco, Inc. (Easton, Md.). Suitable alternative pumps include syringe pumps, such as the 1000D or 260D pumps, which are commercially available from Isco, Inc. (Lincoln, Nebr.).

The SCF pump is in fluid communication with the high-pressure extraction vessel 101 and supplies SCF to the extraction chamber 104. Throughout the instant specification and in the appended claims, the term “SCF” refers to compounds that are in a supercritical state. The SCF is preferably supercritical carbon dioxide (“CO₂”), which is supercritical at a pressure of or greater than 72.9 atmospheres and at a temperature of or greater than 31.1° C. It will be appreciated that some impurities may be removed using compressed or liquefied gases, rather than SCF. Suitable alternative SCF's include, supercritical nitrous oxide, supercritical dimethylether, supercritical straight chain and branched C1-C6-alkanes, supercritical alkenes, supercritical fluoroform, supercritical chlorotrifluoroalkanes, supercritical propylene, supercritical ammonia and combinations thereof. Preferred alkanes include ethane, propane, butane, isopropane, and the like. The SCF must be chosen with reference to its ability to dissolve and extract the impurities from the pharmaceutical composition, either with or without the addition of a modifying co-solvent. In other words, the impurities in the pharmaceutical composition must soluble in the SCF or SCF/modifying co-solvent mixture at conditions wherein the biologically active material is not soluble in the SCF or the mixture.

The pressure inside the extraction chamber is maintained using a backpressure regulator 103. The backpressure regulator 103 is preferably a 26-1700-type regulator, which is commercially available from Tescom, USA (Elk River, Minn.).

Disposed inside each end the extraction vessel are two frits 106 that prevent escape of pharmaceutical composition during extraction of the impurity. The pore size of the frits 106 must be less than the diameter of the pharmaceutical composition.

As a safety precaution, the extraction vessel 101 is preferably fitted with a release valve (not shown). The release valve is preferably a model R3A ¼″ proportional pressure release valve, which is commercially available from Swagelok, Inc. (Solon, Ohio). The release valve is actuated by system pressure acting against a spring, and is capable of reseating.

Operation in accordance with the first embodiment of the invention using apparatus 100 is carried out using the following steps. First the pharmaceutical composition is introduced into the extraction vessel. The heating jacket 105 controls the temperature of the extraction vessel at a predetermined and equilibrated temperature. The supercritical fluid pump supplies a flow of SCF to the extractor chamber 104 at the first end of the extractor at a predetermined constant pressure. The SCF flows through a packed bed or through a fluidized bed of the pharmaceutical composition depending on its flow rate. The SCF dissolves and extracts the impurities from the pharmaceutical composition thereby purifying it in a continuous manner. The rate of extraction of the impurities can be controlled using the pressure and temperature and the flow rate of the SCF. The SCF exiting the extraction chamber is continuously analyzed for the impurity content using a photo diode array (PDA) detector 109. Once the concentration of impurities in the SCF exiting the extraction chamber is below a permissible limit, the SCF flow is terminated and the extraction chamber is depressurized. The purified pharmaceutical composition can then be removed from the extraction chamber.

FIG. 2 schematically illustrates an apparatus 200 for use in carrying out a second embodiment of a method of the invention. The apparatus 200 shown in FIG. 2 includes many of the same components as the apparatus 100 shown in FIG. 1. Accordingly, the same reference numbers are applied to identical components. The primary difference between the apparatus 200 shown in FIG. 2 and the apparatus 100 shown in FIG. 1 is that a plurality of perforated trays 108 are disposed inside extraction chamber 104 of the apparatus 200. The pharmaceutical composition is placed on the perforated trays 108. The SCF percolates through the trays and extracts the impurities from the pharmaceutical composition. In addition, a frit 107 can be used to disperse the SCF into smaller flows within the extraction chamber 104.

Operation in accordance with the second embodiment of the invention using apparatus 200 is carried out using the following steps. First the pharmaceutical composition is loaded onto the perforated trays in the extraction vessel. The heating jacket controls the temperature of the extractor at a predetermined and equilibrated temperature. The supercritical fluid pump supplies SCF to the extractor chamber at the first end of the extractor through a frit at a predetermined constant pressure. The SCF percolates through the trays and extracts the impurities from the pharmaceutical composition placed on the trays. As in the first embodiment of the invention, the rate of extraction of the impurities can be controlled using pressure, temperature and flow rate. Progress of the extraction is monitored using the PDA detector. Once the extraction process is complete, and the impurities in the exit stream are below the permissible levels as indicated by the PDA detector, the SCF flow is terminated and the extraction chamber is depressurized in order to collect the clean product.

In both embodiments of the invention, the operational pressure and temperature conditions are selected such that the impurity present in the pharmaceutical composition is soluble in the SCF, but the biologically active material is not. If the pharmaceutical composition contains multiple impurities, the operational extraction conditions can be changed in order to dissolve and extract each of or parts of the impurities separately. For example, the pressure and temperature of the extraction chamber can be maintained at a first temperature and pressure wherein the SCF dissolves and extracts a first impurity, and then the pressure and temperature of the extraction chamber can be maintained at a second temperature and pressure wherein the SCF dissolves and extracts a second impurity. If a modifying co-solvent is not used, purification of the pharmaceutical composition can be carried out in one step without the use of any organic solvents at mild operating conditions.

Additional advantages and modifications will readily occur to those skilled in the art. Therefore, the invention in its broader aspects is not limited to the specific details and illustrative examples shown and described herein. Accordingly, various modifications may be made without departing from the spirit or scope of the general inventive concept as defined by the appended claims and their equivalents.

What is claimed is:

1. A method of purifying a pharmaceutical composition comprising:
providing a pharmaceutical composition that comprises a biologically active substance and at least a first impurity;
loading the pharmaceutical composition into an extraction chamber;
flowing a supercritical fluid through the extraction chamber in contact the pharmaceutical composition at a first temperature and at a first pressure in which the first impurity is soluble in the supercritical fluid but the biologically active substance is not;
monitoring the concentration of the first impurity dissolved in the supercritical fluid in an exit stream flowing from the extraction chamber;
stopping the flow of supercritical fluid through the extraction chamber when the concentration of the dissolved first impurity in the exit stream is at or below a predetermined level; and
depressurizing the extraction chamber to collect a purified pharmaceutical composition.

2. The method according to claim 1 wherein the pharmaceutical composition further comprises a biodegradable polymer.

3. The method according to claim 1 wherein the supercritical fluid is supercritical carbon dioxide.

4. The method according to claim 1 wherein the pharmaceutical composition further comprises a second impurity and wherein after the first impurity is extracted from the pharmaceutical composition, supercritical fluid is flowed through the extraction chamber in contact the pharmaceutical composition at a second temperature and at a second pressure in which the second impurity is soluble in the supercritical fluid but the biologically active substance is not.

5. The method according to claim 1 wherein the pharmaceutical composition is in the form of discrete particles.

6. The method according to claim 1 wherein the pharmaceutical composition is supported by a tray inside the extraction chamber.

7. The method according to claim 1 wherein the first impurity is selected from the group consisting of residual monomers, unreacted reagents and residual organic solvents.

8. The method according to claim 1 wherein the concentration of the first impurity in the exit stream is monitored using a photo diode array detector.

9. A purified pharmaceutical composition formed in accordance with the method of claim 1.

10. The method according to claim 1 wherein the supercritical fluid flowing through the extraction chamber in contact the pharmaceutical composition is mixed with one or more modifying co-solvents that are at least partially miscible with the supercritical fluid.

11. The method according to claim 10 wherein the pharmaceutical composition further comprises a biodegradable polymer.

12. The method according to claim 10 wherein the supercritical fluid is supercritical carbon dioxide.

13. The method according to claim 10 wherein the pharmaceutical composition further comprises a second impurity and wherein after the first impurity is extracted from the pharmaceutical composition, supercritical fluid is flowed through the extraction chamber in contact the pharmaceutical composition at a second temperature and at a second pressure in which the second impurity is soluble in the supercritical fluid but the biologically active substance is not.

14. The method according to claim 10 wherein the pharmaceutical composition is in the form of discrete particles.

15. The method according to claim 10 wherein the pharmaceutical composition is supported by a tray inside the extraction chamber.

16. The method according to claim 10 wherein the first impurity is selected from the group consisting of residual monomers, unreacted reagents and residual organic solvents.

17. The method according to claim 10 wherein the concentration of the first impurity in the exit stream is monitored using a photo diode array detector.

18. A purified pharmaceutical composition formed in accordance with the method of claim 10.

19. An apparatus for purifying a pharmaceutical composition comprising:
an extraction chamber for receiving a pharmaceutical composition comprising a biologically active substance and at least a first impurity;
a source of supercritical fluid connected to the extraction chamber for flowing supercritical fluid into the extraction chamber;
a valve for adjusting the pressure within the extraction chamber;
a heating jacket disposed on the extraction chamber for adjusting the temperature within the extraction chamber;
a plurality of trays disposed within the extraction chamber for supporting the pharmaceutical composition; and
a photo diode array detector connected to an exit flow from the extraction chamber for monitoring the concentration of the first impurity dissolved in the supercritical fluid.

20. An apparatus for purifying a pharmaceutical composition comprising:
an extraction chamber for receiving a pharmaceutical composition comprising a biologically active substance and at least a first impurity;
a source of supercritical fluid connected to the extraction chamber for flowing supercritical fluid into the extraction chamber;
a source of at least one co-solvent connected to the extraction chamber for mixing with the supercritical fluid and modifying the solvating power of the supercritical fluid;
a valve for adjusting the pressure within the extraction chamber;
a heating jacket disposed on the extraction chamber for adjusting the temperature within the extraction chamber;
a plurality of trays disposed within the extraction chamber for supporting the pharmaceutical composition; and
a photo diode array detector connected to an exit flow from the extraction chamber for monitoring the concentration of the first impurity dissolved in the supercritical fluid/co-solvent mixture.
